(19) United States
(12) Patent Application Publication
(10) Pub. No.: US 2025/0331513 A1
(43) Pub. Date: Oct. 30, 2025

(54) METHODS FOR STORING MRNA COMPOSITIONS

(71) Applicant: eTheRNA immunotherapies NV, Niel (BE)

(72) Inventors: Phillip CHALLIS, Niel (BE); Lubos BAUER, Niel (BE)

(21) Appl. No.: 18/561,862

(22) PCT Filed: May 25, 2022
(86) PCT No.: PCT/EP2022/064237

§ 371 Date: Nov. 17, 2023

(30) Foreign Application Priority Data
May 26, 2021 (EP) 21175871.9

(51) Int. Cl. A01N 1/125; C12P 19/34

(52) U.S. Cl. CPC A01N 1/125; C12P 19/34

(57) ABSTRACT

The present invention relates to the field of RNA-containing compositions, more in particular in the stabilization of in vitro transcribed mRNA-containing compositions for frozen storage. Specifically, the present invention provides a solution for reducing or avoiding the formation of aggregates/precipitates in such RNA-containing compositions, even when being frozen and subsequently thawed. Thereto, the present invention provides means and methods for preventing the formation of aggregates/precipitates in a frozen or thawed RNA-containing composition, by adding a metal binding chelator to said composition prior to freezing said composition.

### CROSS-REFERENCES TO RELATED APPLICATIONS

This application is a national-stage application under 35 U.S.C. § 371 of International Application PCT/EP2022/064237, filed May 25, 2022, which International Application claims benefit of priority to European Patent Application No. 21175871.9, filed May 36, 2021.

### TECHNICAL FIELD

The present disclosure relates to the field of RNA-containing compositions, more in particular in the stabilization of in vitro transcribed mRNA-containing compositions for frozen storage. Specifically, the present disclosure provides a solution for reducing or avoiding the formation of aggregates/precipitates in such RNA-containing compositions, even when being frozen and subsequently thawed. Thereto, the present disclosure provides means and methods for preventing the formation of aggregates/precipitates in a frozen or thawed RNA-containing composition, by adding a metal binding chelator to said composition prior to freezing said composition.

### BACKGROUND

One of the major challenges in the field of in vivo applications of mRNA/RNA-containing compositions is the stability of the RNA. By nature double-stranded DNA is much more stable compared to single-stranded which is much more flexible and can form weak internal bonds. Also RNA has a hydroxyl group on the second carbon of the ribose sugar which increases the likelihood of breakage of the sugar-phosphate backbone. In addition, RNA is very sensitive to degradation by endogenous ribonucleases, which can however, be rapidly inactivated upon freezing RNA samples. Accordingly, RNA is typically frozen for prolonged storage.

Whereas, in vitro transcribed RNA may be used directly for further processing or purification, it may sometimes be advantageous to freeze the RNA first, such as in the event that the further processing takes place in another facility; or for accumulating larger batches for further processing. However, in such instance of freezing the RNA after in vitro transcription but prior to further processing, the inventors of the present application identified that low temperature storage of particular mRNA molecules resulted in aggregation thereof after thawing the samples. Obviously, aggregation of mRNA molecules after thawing is highly unwanted, specifically where further processing/purification is still required. For example, precipitation is believed to be the main cause of loss of in vitro transcribed mRNA, when samples are subjected to subsequent column purification steps. Accordingly, it was an object of the present disclosure to provide a solution to this problem.

The observation of formation of aggregates was particularly made in in vitro transcribed mRNA samples containing a CAP-1 structure, most profoundly in the presence of divalent metal ions. Such metal ions are typically present during the in vitro transcription process as a co-factor of enzymes used therein.

The inventors of the present disclosure unexpectedly found that metal ions, in particular divalent metal ions such as Mg²⁺ was one of the components that were involved in the process of precipitation of the mRNA_IVT mix after thawing the mix after it has been stored at temperatures below 0° C. The addition of a metal binding chelator such as EDTA to sequester metal ions such as Mg²⁺ effectively resolved the issue of precipitation in mRNA molecules containing a CAP-1 structure. The inventors show that precipitation occurs under at least 3 circumstances: First, mRNA samples containing a CAP-1 structure show precipitation. Second, precipitation only occurs in samples thawed after a storage temperature below 0° C. Third, precipitation occurs in samples containing metal ions, such as Mg²⁺ cations.

Accordingly, the present disclosure provides a solution for reducing or avoiding the formation of aggregates/precipitates in such RNA-containing compositions, even when being frozen and subsequently thawed. The presence of a metal binding chelator such as EDTA at the end of the IVT reaction has at least three major benefits. In addition to (i) maintaining the charge and conformational homogeneity of RNA and (ii) the prevention of formation of aggregate in certain conditions, the presence of a metal binding chelator (iii) prevents RNA degradation since metal-dependent enzymes acting as nucleases become deactivated. The ability of EDTA to prevent aggregation and RNA degradation might play a crucial role in the scenario where long-term storage of the mRNA IVT_mix will be required.

### SUMMARY

In a first aspect, the present disclosure relates to a method for reducing and/or preventing the formation of aggregation in a frozen or thawed composition comprising in vitro transcribed mRNA having a 5′ CAP-1 structure; said method comprising: adding a metal binding chelator to said composition prior to freezing said composition.

In a specific embodiment of the present disclosure, said composition further comprises divalent metal ions.

In another specific embodiment of the present disclosure, said metal binding chelator is capable of binding divalent metal ions.

In a particular embodiment, said divalent metal ions are selected from the list comprising: Ca²⁺ ions, Cu²⁺ ions, Fe²⁺ ions, Zn²⁺ ions, and Mg²⁺ ions; optionally Mg²⁺ ions.

In yet another particular embodiment, said metal binding chelator is used at an equimolar concentration (mol dm 3) or higher concentration than the concentration of said divalent metal ions in said composition.

In a specific embodiment of the present disclosure, said metal binding chelator is selected from the list comprising: BAPTA (1,2-Bis(2-aminophenoxy) ethane-N,N,N′,N′-tetraacetic acid), DFOA (Deferoxamine Mesylate), Dimethoxynitrophenamine (1-(2-Nitro-4,5-dimethoxyphenyl)-1,2-diaminoethane-N,N,N′,N′-tetraacetic Acid), EDTA (ethylenediaminetetraacetic acid), sodium calcium EDTA, tetrasodium EDTA, EGTA (ethylene glycol-bis(β-aminoethyl ether)-N,N,N′,N′-tetraacetic acid), CDTA (1,2-cyclohexylenedinitrilo)tetraacetic acid), DPTA (diethylenetriaminepentaacetic acid), PIH (pyridoxal isonicotinoyl hydrazone), TPEN (N′-Tetrakis(2-pyridylmethyl)ethylenediamine).

In a particular embodiment of the present disclosure, said frozen or thawed composition will be or has been frozen at −20° C. or less.

In another particular embodiment, said thawed composition will or has been thawed at about and between 20 and 30° C.

In a further aspect, the present disclosure also provides the use of a metal binding chelator for reducing and/or preventing the formation of aggregation in a frozen or thawed composition comprising in vitro transcribed mRNA molecules having a CAP-1 structure.

### DETAILED DESCRIPTION

As already detailed herein above, the present disclosure relates to stabilization of in vitro transcribed mRNA-containing compositions for frozen storage. In particular, it relates to the avoidance or reduction of precipitation in frozen samples, thereby significantly increasing the yield of mRNA after subsequent purification such as column precipitation. In particular, after IVT (in vitro transcription) it may be beneficial to store the obtained mRNA compositions for a certain period of time prior to further processing, such as where further processing is done at a different location/facility. However, due to the nature of mRNA, such storage is optionally done at reduced temperatures of −20° C. or less. The inventors have found, though that storage of IVT mRNA mixes at reduced temperatures resulted in the formation of aggregates/precipitates, which are obviously undesired for further processing. Thereto, the inventors have set-up a research program to identify the causes for the formation of such precipitates, as well as solutions to avoid/reduce such precipitates. Details thereof may be found in the examples part herein below.

Based on these observations, a first aspect of the present disclosure relates to a method for reducing and/or preventing the formation of aggregation in a frozen or thawed composition comprising in vitro transcribed mRNA having a 5′ CAP-1 structure; said method comprising: adding a metal binding chelator to said composition prior to freezing said composition.

In the context of the present disclosure, the terms ‘reducing’ or alternatively ‘to reduce’ are meant to be to ‘lessen’, to ‘decrease’, to ‘minimize’, or to ‘diminish’ the formation of aggregation in said samples. Accordingly, where a sample would under normal circumstance contain a particular amount of aggregates after being frozen, the term ‘reducing’ means that said amount of aggregates is lower when subjecting said sample to the method of the present disclosure. In particular, the amount of aggregates is optionally be reduced by at least 10%, such as at least 20%, at least 30%, at least 40%, at least 50%, at least 60%, at least 70%, at least 80%, at least 90%, when compared to untreated samples.

In the context of the present disclosure, the term ‘preventing’ or alternatively ‘to prevent’ are meant to be to ‘stop’, to ‘avert’, to ‘arrest’, to ‘block’, or to ‘halt’, completely (i.e. 100%) or at least significantly the formation of aggregation in said samples. Wherein significantly is meant to be at least for 95%, such as at least for 96%, at least for 97%, at least for 98%, at least for 99%, when compared to untreated samples.

The amount of aggregates may be determined by visual inspection of the samples, or alternatively by experimental determination using suitable techniques. For example, UV spectrum analysis may be used for measuring aggregation by determining a change in UV spectrum at particular wavelengths (e.g. 600 nm) as a measure of precipitate formation/turbidity. The underlying principle for a signal measurement is the detection of light scattering at particular wavelengths, wherein the light transmission through the sample acts as a turbidimeter, so that the electric signal is weaker in a cloudy (precipitated sample) than in a precipitate-free sample.

In the context of the present disclosure the term ‘the formation’ is meant to be ‘the emergence’, ‘the development’, ‘the origination’, or ‘the generation’ of aggregates in said samples. Specifically, samples obtained after in vitro transcription are typically free from aggregates, while we have identified that the presence of particular components may results in the formation of such aggregates upon a frozen/thaw cycle.

In the context of the present disclosure, the terms ‘aggregates’ or alternatively ‘aggregation’ are meant to be ‘agglomeration’, ‘clustering’, ‘clumping’, or ‘accumulation’ of components present in the composition, such that said components are in such close proximity to each-other that they form aggregates, clusters, clumps, . . . which may or may not be big enough for visual inspection, such as in the form of a cloudy appearance of the samples or even bigger individually appreciable flocks of aggregates. Said aggregation may also result in the formation of precipitates which are big enough to form a layer of aggregates at the bottom of the test tubes comprising said compositions.

In the context of the present disclosure, the term ‘frozen’ or alternatively ‘to freeze’ is meant to be ‘to chill’, ‘to ice’, or ‘to reduce the temperature’ below 0° C. Optionally the compositions of the present disclosure are frozen at temperature of about −20° C. or lower. In particular embodiments, the compositions of the present disclosure may be frozen at temperature of about −80° C.

In the context of the present disclosure, the term ‘thawed or alternatively ‘to thaw is meant to be ‘to defrost, ‘to deice’, or ‘to increase the temperature’ to about or above 0° C. Optionally the compositions of the present disclosure are thawed at room temperature such as about 20° C.-25° C. or higher. In particular embodiments, the compositions of the present disclosure may be thawed at temperature of about 30° C., such as about 35° C., about 40° C., about 45° C., about 50° C., about 55° C., about 60° C., about 65° C., about 70° C., or about 75° C.

It was particularly found that a single cycle of freezing and thawing results in the formation of aggregates in in vitro transcribed mRNA samples, especially in those having a 5′ CAP-1 structures. Evidently, multiple cycles of freezing and thawing only worsens the observed effect.

In the context of the present disclosure, the term ‘metal chelator’ is meant to be a molecule capable of bonding to metal ions. Accordingly, metal chelators can be used for capturing metal ions in a composition such that they can no longer interact with negatively charged ions in said composition.

The observed aggregation was specifically profound in compositions comprising positive metal ions. In the context of the present disclosure, the term ‘positive metal ion’ is meant to be a cation, in particular a metal having a net electrical charge being positive. A cation is a positively charged ion with fewer electrons than protons, i.e. is has lost electrons. In the context of the present disclosure, aggregation was most profound with a double charged (or divalent) metal ions or cations. These are ions having lost 2 electrons and are typically annotated as follows M²⁺; wherein ‘M’ represents a metal atom and ‘²⁺’ represent the divalent charge.

Accordingly, in a specific embodiment of the present disclosure, the composition may further comprise positive metal ions; in particular divalent metal ions. In a particular embodiment, said divalent metal ions are selected from the list comprising: Ca²⁺ ions, Cu²⁺ ions, Fe²⁺ ions, Zn²⁺ ions, and Mg²⁺ ions; optionally Mg²⁺ ions. Alternatively suitable divalent metal ions may for example be Ba²⁺ ions, Be²⁺ ions, Pb²⁺ ions, Mn²⁺ ions, and Sn²⁺ ions.

Accordingly, in the context of the present disclosure, it is advantageous to use a metal binding chelator capable of binding divalent metal ions. In a specific embodiment of the present disclosure, said metal binding chelator may be from the list comprising: BAPTA (1,2-Bis(2-aminophenoxy) ethane-N,N,N′,N′-tetraacetic acid), DFOA (Deferoxamine Mesylate), Dimethoxynitrophenamine (1-(2-Nitro-4,5-dimethoxyphenyl)-1,2-diaminoethane-N,N,N′,N′-tetraacetic Acid), EDTA (ethylenediaminetetraacetic acid), sodium calcium EDTA, tetrasodium EDTA, EGTA (ethylene glycol-bis(β-aminoethyl ether)-N,N,N′,N′-tetraacetic acid), CDTA (1,2-cyclohexylenedinitrilo)tetraacetic acid), DPTA (diethylenetriaminepentaacetic acid), PIH (pyridoxal isonicotinoyl hydrazone), TPEN (N′-Tetrakis(2-pyridylmethyl)ethylenediamine).

In particular, it is known that one molecule of a metal chelator such as EDTA is capable of chelating one metal ion. Therefore, in order to capture sufficient amounts of divalent metal ions from the compositions in order to maximally reduce aggregation, it is advantageous to use the metal binding at an equimolar concentration (mol dm⁻³) or higher than the equimolar concentration of said divalent metal ions in said composition.

The addition of metal chelators potentially has three benefits. Firstly, it will stop enzymatic reactions that require the presence of metal ions as a cofactor, and secondly it will chelate metal ions thereby preventing the formation of the aggregate. Lastly, the addition of a metal chelator will help to maintain the homogeneity of the RNA since divalent ions contribute to non-specific interactions that create charge and conformational heterogeneity in RNA.

In a particular embodiment, in vitro transcribed mRNA molecules might have a 5′ CAP-1, 5′ CAP-2, 5′ m6Am structure, or derivatives thereof. The eukaryotic 5′ cap consists of a 7-methylguanosine (m7G) connected by a triphosphate bridge to the first nucleotide, forming a structure known as ARCA cap analog (5′ CAP-0 analog). In the context of the present disclosure, the term “5′ CAP-1” is meant to be a CAP-0 structure with an additional methyl group (2′ mono methylated) at the second carbon of the ribose sugar of the first cap-proximal nucleotide, such as represented herein below:

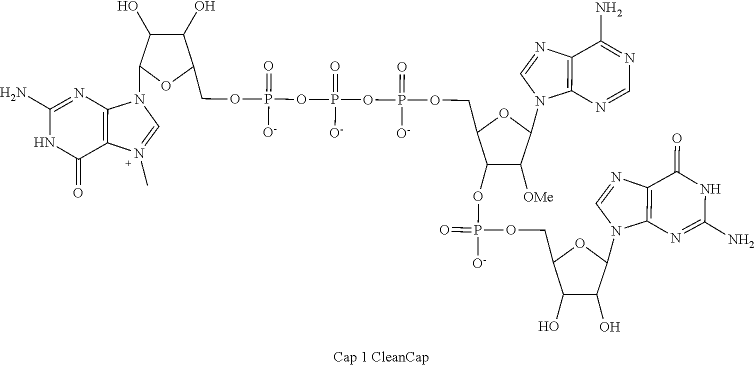

In the context of the present disclosure, the term “5′ CAP-2” is meant to be a CAP-1 structure with an additional methyl group (2′ dimethylated) at the second carbon of the ribose sugar of the second cap-proximal nucleotide. In the context of the present disclosure, the term “5′ m6Am” structure is meant to be a CAP-1 structure wherein the first nucleotide is an adenosine with a methyl group at the sixth nitrogen forming N6-methyladenosine (m6Am). As used herein, capping may involve a capping strategy during the mRNA manufacturing. In a specific embodiment, capping may refer to ‘co-transcriptional capping’ wherein a cap analog is incorporated during transcription. For example, CleanCap® technology is a proprietary, co-transcriptional 5′ capping solution that generates a natural Cap 1 structure. In another embodiment, capping may refer to ‘posttranscriptional capping’ wherein a cap analog is incorporated after the mRNA synthesis using an enzyme-based method.

In a further aspect, the present disclosure also provides the use of a metal binding chelator for reducing and/or preventing the formation of aggregation in a frozen or thawed composition comprising in vitro transcribed mRNA molecules having a CAP-1 structure.

### EXAMPLES

Previous observations have shown that an in vitro transcribed mRNA mix (i.e. IVT_mRNA mix) containing CleanCap® (i.e. a type of 5′ CAP-1 structure) has been precipitating during or after the transfer on cool packs or after storage at −20° C. This precipitation is believed to be the main cause of loss of in vitro transcribed mRNA, specifically when samples are subjected to subsequent column purification steps. Accordingly, the examples detailed herein after illustrate the conditions leading to such precipitation and solutions for avoiding or at least reducing precipitation, thereby increasing the mRNA yield even after further purification steps.

### Example 1: Precipitation of In Vitro Transcribed mRNA with 5′CAP-1 after Storage

In this experiment, it was investigated whether the sample precipitation can be avoided by diluting the IVT_mRNA mix with 1.5 volume of WFI (water for injection) right after the DNaseI treatment, which corresponds with a 2.5-fold dilution of the IVT_mRNA mix. Furthermore, the protocol aimed to examine the impact of storage conditions (at 4° C. and −20° C. for 24 hours) and subsequent thawing of the IVT_mRNA mix on the integrity of mRNA. Additionally, the plausible root cause, that the mRNA was co-precipitated in the IVT_mRNA mix was verified. The protocol was applied on a small scale production of four different IVT_mRNA mixes encoding for human gp100, huCD40L, huCD70 and TLR4ca. These four cases represent mRNAs of different length, GC content and minimal free energy of their secondary structure. As a negative control WFI was used instead of the linearized DNA template.

### Experimental Set-Up & Results

0.8 ml of unmodified mRNA encoding for gp100, huCD40L, huCD70 and TLR4caco with a CleanCap® structure at the 5′ extremity of the mRNA were produced. As a negative control, a 0.8 ml reaction where the DNA template is replaced with WFI is performed. Right after DNase treatment (800 U/ml for 30 min), each of the four IVT_mRNA mixes (0.8 ml) and the control reaction are thoroughly mixed and divided into Eppendorf tubes (20 samples of 0.2 ml fraction each).

0.2 ml of each IVT_mRNA mix, and the control mix are kept at 4° C. for 24 hours

0.2 ml of each IVT_mRNA mix, and the control mix are immediately diluted with 1.5 vol. of WFI (that equals to 0.3 ml WFI) and kept at 4° C. for 24 hours.

0.2 ml of each IVT_mRNA mix, and the control mix are kept at −20° C. for 24 hours

0.2 ml of each IVT_mRNA mix, and the control mix are immediately diluted with 1.5 vol. of WFI (that equals to 0.3 ml WFI) and kept at −20° C. for 24 hours.

After 24 hours the samples were placed into a block heater set to 30° C. for 10 min. In the next step the samples were brought to room temperature and visually evaluated for signs of precipitation. The presence of the precipitate was photo-documented (Table 1).

TABLE 1
Visual evaluation of IVT_mRNA mix precipitation.

| | | | | | | −CTRL |
|---|---|---|---|---|---|---|
| Storage temp. | Dilution | gp100 | huCD40L | huCD70 | TLR4 | (WFI) |
| 4° C. | none | cloudy | − | − | − | − |
| | 2.5x | Slightly | − | − | − | − |
| | | cloudy | | | | |
| −20° C. | none | + | − | − | − | + |
| | 2.5x | + | + | + | + | + |

− no precipitation
+ precipitation

As detailed in Table 1, no visual sign of sedimented precipitate was observed in samples stored at 4° C. regardless of the dilution. The only exception was gp100 that was cloudy at both undiluted and diluted state at 4° C. Surprisingly, in all samples that were diluted (2.5×) and stored at −20° C. a sedimented precipitate was formed. Two of the undiluted samples stored at −20° C. (gp100 and the WFI negative control) formed sedimented precipitates. Based on the results, it can be concluded that a simple dilution of the IVT_mRNA mix in WFI is not enough to resolve the issue of precipitation upon storage at −20° C. Storage at 4° C. looks more promising since no sedimented precipitate was detected at this temperature. However, the main disadvantage of storage at 4° C. is the higher risk of RNA degradation.

As in Table 1, the only condition where all the samples formed a precipitate was after dilution and storage at −20° C. These sedimented precipitates were centrifuged (13000 rpm for 10 min) and dissolved in WFI for further analysis. The amount of RNA in the dissolved precipitate was determined (Table 2). The highest amount of RNA was found in the gp100 sample. This RNA was found to be most susceptible to form precipitates after storage. It is important to note that these samples might still contain some free NTPs since no purification was performed on them. This is reinforced by the presence of NTPs in the negative control reaction.

TABLE 2
Amount of RNA (μg) in the precipitate.

| | | | | | | −CTRL |
|---|---|---|---|---|---|---|
| Storage temp. | Dilution | gp100 | huCD40L | huCD70 | TLR4 | (WFI) |
| −20° C. | 2.5x | 102.7 | 17.9 | 19.5 | 21.7 | 8.4 |

### Example 2: Causes of Precipitation in Frozen/Thawed IVT_mRNA Mix

Since a 2.5× dilution of IVT_mRNA mix did not inhibit the precipitation of samples after storage at −20° C. (see example 1) an investigation of the root cause of precipitation was started. It is important to note that the precipitation only occurred with mRNA IVT mix for which a CleanCap® (i.e. CC) cap analog (5′ CAP-1 analog) was used in the reaction. The legacy IVT procedure utilizing the ARCA cap analog (5′ CAP-0 analog) did not show any signs of precipitation after storage (data not shown). Since the negative control without DNA template and rationally without RNA also formed a precipitate after storage at −20° C. and subsequent thawing it is reasonable to assume that the nucleic acids themselves are not responsible for initiating the precipitation. The other components that are in the reaction are as follows: T7 reaction buffer, NTP/cap mix (ARCA or CleanCap®), and T7 enzyme mix (T7 polymerase, RNasin Ribonuclease inhibitor and Pyrophosphatase). To find out which of the components are responsible for the precipitation, mock IVT mixes devoid of components where prepared. The mock_IVT mix can be defined as: IVT_mix without the DNA template and proteins used in the process of in vitro transcription. The reaction is not incubated at 37° C. The first investigation focused on the T7 reaction buffer and the NTP/CleanCap or ARCA mix. The T7 reaction buffer contains the following components: HEPES pH 7.8 (452 mM), MgCl₂ (120 mM), Spermidine (10 mM), Dithiothreitol (DTT) (200 mM) and WFI. The NTP/CleanCap mix contains the following components: ATP (20 mM), CTP (20 mM), UTP (20 mM), GTP (20 mM), and CleanCap (20 mM). The NTP/ARCA mix contains the following components: ATP (12 mM), CTP (12 mM), UTP (12 mM), GTP (2.4 mM), ARCA (9.6 mM), and WFI.

The first investigation was conducted on mock IVT_mixes devoid of the template DNA and the T7 enzyme mix in the presence or absence of NTP/cap. WFI was used to substitute the components absent from the reaction. The mock IVT_mixes were prepared in accordance with Table 3.

TABLE 3
Components in the mock IVT_mix.

| | IVT_mRNA mix | | | | | |
|---|---|---|---|---|---|---|
| | 1 | 2 | 3 | 4 | 5 | 6 |
| WFI | 160 μl | 160 μl | 110 μl | 90 μl | 110 μl | 90 μl |
| Lin_pDNA | — | — | — | — | — | — |
| T7 buffer 5x | 40 μl | 40 μl | 40 μl | 40 μl | 40 μl | 40 μl |
| NTP/mix CC | — | — | 50 μl | — | 50 μl | |
| NTP/mix | — | — | — | 70 μl | — | 70 μl |
| ARCA | | | | | | |
| T7 enzyme mix | — | — | — | — | — | — |
| Total | 200 μl | 200 μl | 200 μl | 200 μl | 200 μl | 200 μl |

After assembly, no dilution was performed, and the reactions were stored at −20° C. for 20 hours and thawed at 30° C. for 10 minutes. For comparison, two different batches of T7 reaction buffers were used. In eppendorfs 1, 3 and 4 a first batch of T7 reaction buffer was used. In eppendorfs 2, 5 and 6 a second batch of T7 reaction buffer was used. The results of these experiments are detailed in Table 4.

TABLE 4
Visual evaluation of IVT_mRNA precipitation in
mixes of Table 3 (−) no prec. (+) prec.
IVT_mRNA mix

| 1 | 2 | 3 | 4 | 5 | 6 |
|---|---|---|---|---|---|
| − | − | + | − | + | − |

Based on the results presented in Table 4, it can be concluded that the precipitation occurs only in the reactions where the NTP/CleanCap® mix (i.e. NTP/CC mix) was present. No precipitation was found in the T7 reaction buffers and in the T7 reaction buffers supplemented with the NTP/ARCA mix. Since the NTP/CC mix only forms precipitates in the presence of the T7 reaction buffer, it is reasonable to assume that the precipitation was caused by some of the components of the T7 reaction buffer in the presence of the CleanCap® cap analog. At this point it is straightforward to rule out the possibility that the NTPs are responsible for the precipitation. To address the question which component of the T7 reaction buffer was responsible for the precipitation in the presence of the CleanCap® a series of T7 reaction buffers were prepared. Each of the components was systematically left out from the formulation and was substituted with WFI. Four buffers were prepared: T7 buffer with all components; T7 buffer—DTT; T7 buffer—spermidine; T7 buffer—MgCl₂. Subsequently, these T7 reaction buffers where used to prepare mock IVT_mixes as detailed in Table 5. After assembly, no dilution was performed, and the mixtures were stored at −20° C. for 72 hours. Subsequently, the samples were thawed at 30° C. for 10 minutes.

TABLE 5
Components in the mock IVT_mix.

| | IVT_mRNA mix | | | | |
|---|---|---|---|---|---|
| | 1 | 2 | 3 | 4 | 5 |
| WFI | 110 | 90 | 110 | 110 | 110 |
| Lin_pDNA | — | — | — | — | — |
| T7 reaction | 40 μl | 40 μl | 40 μl − | 40 μl − | 40 μl − |
| buffer 5x | complete | complete | DTT | spermidine | MgCl₂ |
| NTP/mix CC | 50 | — | 50 | 50 | 50 |
| NTP/mix | — | 70 | — | — | — |
| ARCA | | | | | |
| T7 enzyme mix | — | — | — | — | — |
| Total | 200 μl | 200 μl | 200 μl | 200 μl | 200 μl |

Results presented in Table 6 confirmed previous observations.

TABLE 6
Visual evaluation of IVT_mRNA precipitation in
mixes of Table 5 (−) no prec. (+) prec.

| | | | | | |
|---|---|---|---|---|---|
| IVT_mRNA mix | 1 | 2 | 3 | 4 | 5 |

| | | | | |
|---|---|---|---|---|
| + | − | + | + | − |

Once again when NTP/CC and NTP/ARCA are compared head to head (reaction 1 and 2) the precipitate was detected only in the formulation with CleanCap®. Only the T7 reaction buffer lacking MgCl₂ (reaction 5) showed no signs of precipitation. To conclude, this result suggests that MgCl₂ with the CleanCap® analog are the key components responsible for the formation of the precipitate.

To further investigate the role of MgCl₂ in the process, it was important to find out, which ion Mg²⁺ or Cl⁻ was responsible for the precipitation in the presence of the CleanCap®. For this reason, 5×T7 reaction buffers where the 120 mM MgCl₂ was substituted for 120 mM LiCl or 120 mM NaCl were prepared. Li⁺ and Na⁺ were supposed to substitute for the Mg²⁺ cation and in case of no precipitation the Cl-anion would be ruled out as a possible cause of precipitation. Rationally, this would confirm the role of Mg²⁺ in the process. In this case the mock IVT_mixes were stored at both 4° C. and −20° C. for 24 hours and thawed at 30° C. for 10 minutes. The formulations of the mock IVT_mixes where the same as used in the experiments above. The only change was the substitution of MgCl₂ for LiCl or NaCl (Table 7)

TABLE 7
Components in the mock IVT_mix.

| | IVT_mRNA mix | | | | | |
|---|---|---|---|---|---|---|
| | 1 | 2 | 3 | 4 | 5 | 6 |
| WFI | 110 μl | 110 μl | 110 μl | 90 μl | 90 μl | 90 μl |
| Lin_pDNA | — | — | — | — | — | — |
| T7 reaction | 40 μl | 40 μl | 40 μl | 40 μl | 40 μl | 40 μl |
| buffer 5x | MgCl₂ | LiCl | NaCl | MgCl₂ | LiCl | NaCl |
| NTP/mix CC | 50 μl | 50 μl | 50 μl | — | — | — |
| NTP/mix | — | — | — | 70 μl | 70 μl | 70 μl |
| ARCA | | | | | | |
| T7 enzyme mix | — | — | — | — | — | — |
| Total | 200 μl | 200 μl | 200 μl | 200 μl | 200 μl | 200 μl |

The results presented in Table 8 are in line with previous findings.

TABLE 8
Visual evaluation of IVT_mRNA precipitation in
mixes of Table 7 (−) no prec. (+) prec.

IVT_mRNA mix

| | 1 | 2 | 3 | 4 | 5 |
|---|---|---|---|---|---|
| 4° C. | − | − | − | − | − |
| −20° C. | + | − | − | − | − |

No precipitation was observed at 4° C. regardless of the presence of MgCl₂, LiCl or NaCl. At −20° C. only the mock IVT_mix with MgCl₂+NTP/CC formed the precipitate (Reaction 1). This suggests that the Cl⁻ is not causing the precipitation. Accordingly, Mg²⁺ and the CleanCap® are identified as the key components causing the precipitation. Surprisingly, a freezing and thawing cycle is also required for the precipitation to occur.

### Example 3: Solutions for Reducing Precipitation in Frozen/Thawed IVT_mRNA Mix

Accordingly, as detailed in example 2, it was identified that the combination of Mg²⁺ and the CleanCap® are key components causing the precipitation. Therefore, in this example we tested for solutions to avoid such precipitation, by chelating the Mg²⁺ with increasing amounts of Ethylenediaminetetraacetic acid (EDTA). EDTA is a chelating agent widely used in medicine and molecular biology to sequester divalent and trivalent metal ions such as calcium, magnesium, lead and iron. It may further be advantageous to use EDTA in view of its ability to prevent DNA and RNA degradation as metal-dependent enzymes acting as nucleases are likely to become deactivated.

After formulation, the mock IVT_mixes were diluted 2-fold with increasing concentrations of WFI-EDTA solutions (Table 9) and stored at both 4° C. and −20° C. for 24 hours and thawed at room temperature for 10 minutes. Room temperature was chosen instead of 30° C., because it was observed that the precipitate tends to partially dissolve at elevated temperatures.

TABLE 9
Concentration of EDTA in mock IVT_mixes after dilution.

| | IVT_mRNA mix | | | | | | |
|---|---|---|---|---|---|---|---|
| | 1 | 2 | 3 | 4 | 5 | 6 | 7 |
| EDTA | — | — | 6 mM | 12 mM | 24 mM | 48 mM | 240 mM |
| Dilution | — | 2x | 2x | 2x | 2x | 2x | 2x |

Again, it was confirmed that no precipitation was observed at 4° C. regardless of the presence of ARCA or CleanCap (Table 10).

TABLE 10
Visual evaluation of IVT_mRNA precipit. in mixes
of Table 9 (−) no prec. (+) prec.

| IVT_mRNA mix | 1 | 2 | 3 | 4 | 5 | 6 | 7 |
|---|---|---|---|---|---|---|---|
| 4° C. - | − | − | − | − | − | − | − |
| NTP/ARCA | | | | | | | |
| 4° C. - NTP/CC | − | − | − | − | − | − | − |
| −20° C. - | − | − | − | − | − | − | − |
| NTP/ARCA | | | | | | | |
| −20° C. - NTP/CC | + | + | − | − | − | − | − |

Furthermore, it was confirmed that the precipitation seems to be exclusive only for the condition where CleanCap® was used and the mixture was stored at −20° C. No aggregation was observed when CleanCap® was substituted for ARCA. Visual investigation suggests that as little as 6 mM EDTA (Reaction 3) can stop the precipitation. Although, there was no visible precipitate after the addition of 6 mM EDTA, it is still possible that the precipitation might occur to some extent after prolonged storage time. In a hold-time study wherein IVT-mix with EDTA was stored at −20° C. for up to 3 months, no degradation of RNA was observed (data not shown). After a 2× dilution the 24 mM MgCl₂ was diluted to 12 mM. Based on this assumption, 12 mM EDTA should chelate all the Mg²⁺ in the solution. Since these experiments were performed on mock IVT_mixes without the presence of nucleic acids and proteins, a higher concentration of EDTA may be used in practice, to completely avoid the possibility of precipitate formation.

### CONCLUSION

To summarize, the results suggest that three conditions must be fulfilled for the precipitation to occur. The first condition was the use of the CleanCap® analog. No precipitation was observed during the investigation when the ARCA cap analog was used. The second condition was the presence of Mg²⁺ cation in the reaction. Substitution of the Mg²⁺ for Na⁺ or Li⁺ was not accompanied with precipitation. The third condition was the storage temperature at −20° C. No precipitation was observed at 4° C. The underlying solution to the precipitation of the mRNA_IVT mix after storage emerged after the realization that Mg²⁺ was one of the components that were involved in the process. The addition of EDTA as a chelating agent to sequester Mg²⁺ effectively resolved the issue of precipitation. The ability of EDTA to prevent RNA degradation as metal-dependent enzymes acting as nucleases become deactivated might play a crucial role in the scenario where long-term storage of the mRNA IVT_mix will be required.

1-9. (canceled)

10. A method for reducing and/or preventing the formation of RNA aggregates, the method comprising:
preparing a composition comprising RNA and a metal binding chelator, wherein the RNA is in vitro transcribed and has a 5′ CAP-1 structure; and
freezing the composition.

11. The method according to claim 10, wherein the composition further comprises divalent metal ions.

12. The method according to claim 10, wherein the metal binding chelator is capable of binding divalent metal ions.

13. The method according to claim 11, wherein the divalent metal ions are selected from: Ca²⁺ ions, Cu²⁺ ions, Fe²⁺ ions, Zn²⁺ ions, or Mg²⁺ ions.

14. The method according to claim 11, wherein the divalent metal ions are Mg²⁺ ions.

15. The method according to claim 11, wherein the concentration of metal binding chelator is equimolar to the concentration of divalent metal ions.

16. The method according to claim 11, wherein the concentration of metal binding chelator is higher than equimolar to the concentration of divalent metal ions.

17. The method according to claim 10, wherein the metal binding chelator is selected from BAPTA (1,2-Bis(2-aminophenoxy) ethane-N,N,N′,N′-tetraacetic acid), DFOA (Deferoxamine Mesylate), Dimethoxynitrophenamine (1-(2-Nitro-4,5-dimethoxyphenyl)-1,2-diaminoethane-N,N,N′,N′-tetraacetic Acid), EDTA (ethylenediaminetetraacetic acid), sodium calcium EDTA, tetrasodium EDTA, EGTA (ethylene glycol-bis(β-aminoethyl ether)-N,N,N′,N′-tetraacetic acid), CDTA (1,2-cyclohexylenedinitrilo)tetraacetic acid), DPTA (diethylenetriaminepentaacetic acid), PIH (pyridoxal isonicotinoyl hydrazone), or TPEN (N′-Tetrakis(2-pyridylmethyl)ethylenediamine).

18. The method according to claim 10, wherein freezing the composition comprises freezing the composition at a temperature of −20° C. or less.

19. The method according to claim 10, further comprising thawing the composition.

20. The method according to claim 19, wherein thawing the composition comprises thawing the composition at a temperature from about 20° C. to about 30° C.
